SEQUENCE LISTING (1 sequence)

SEQ ID NO: 1 - protein, 144 aa, Homo sapiens
VTRQMCIYTNPTSCNEIYGKFSSAYLACDGKQMEIITLLNPSLISGDEWQWSGNTPIHVLGMWHYSKVLKLLDQDEKSYVKLLSANQSMCSAQSEYWNKSEDFFQYCNNKYCIWNKPCPGCYSETLPIQVEQNFLSNLFVASWI

(19) United States
(12) Patent Application Publication
(10) Pub. No.: US 2022/0154144 A1
(43) Pub. Date: May 19, 2022

(54) ARTIFICIAL ANTIGEN PRESENTING CELLS COMPRISING LIGANDS FOR NKG2D FOR EXPANDING IMMUNE CELLS FOR IMMUNOTHERAPY

(71) Applicant: H. LEE MOFFITT CANCER CENTER AND RESEARCH INSTITUTE, INC., Tampa, FL (US)

(72) Inventor: Marco DAVILA, Tampa, FL (US)

(21) Appl. No.: 17/606,489

(22) PCT Filed: Apr. 27, 2020
(86) PCT No.: PCT/US2020/030042

§ 371 Date: Oct. 26, 2021

Related U.S. Application Data
(60) Provisional application No. 62/839,057, filed on Apr. 26, 2019.

(51) Int. Cl. C12N 5/0783; A61K 35/17

(52) U.S. Cl. CPC C12N 5/0636; A61K 35/17

(57) ABSTRACT

Disclosed herein are methods of expanding immune cells for immunotherapy and/or increasing the purity of a population of CAR T cells using artificial antigen presenting cells (aAPCs) having on their surface NKG2D ligand (such as for example MICA, MICB, RAET1E/ULBP4, RAET1G/ULBP5, RAET1 H/ULBP2, RAET1/ULBP1, RAET1UULBP6, and/or RAET1N/ULBP3 as well as mouse ligands H60, MULT-1, and Rae-1) and/or antibodies that bind NKG2D (including, but not limited to antibody fragments, such as, for example, F (ab′)2, Fab′, Fab, and/or scFv).

### CROSS REFERENCE TO RELATED APPLICATIONS

This application claims priority to U.S. Patent Application No. 62/839,057, filed on Apr. 26, 2019, which is incorporated by reference herein.

### BACKGROUND

Adoptive cell therapy (ACT) using chimeric antigen receptor (CAR) T cells, tumor infiltrating lymphocytes (TIL), and/or marrow-infiltrating lymphocytes (MIL) can lead to positive, objective, and durable responses in cancer patients. However, this therapy can involve sophisticated cell processing techniques and equipment. These procedures have introduced technical, regulatory, and logistic challenges to the successful use of CAR T cells, MIL, TIL as a biological therapy. Accordingly, there is a need in the art for improved methods for growing CAR T cells, MIL, and/or TIL for use in adoptive cell therapy.

### SUMMARY

Disclosed herein are methods of expanding immune cells for immunotherapy using artificial antigen presenting cells (aAPCs) having on their surface ligands for natural-killer group 2, member D (NKG2D) (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or antibodies that bind NKG2D (including, but not limited to antibody fragments, such as, for example, F(ab′)2, Fab′, Fab, and/or scFv) and/or ligands that bind molecules of both the T cell activation pathway and T cell co-stimulation pathway. The disclosed aAPCs can also express cell surface proteins that bind molecules of the T cell activation and co-stimulation pathway. For example, anti-CD3 scFv on the surface of the aAPCs can bind and activate T cells, while anti-CD28 scFv and 4-1BBL on the surface of the aAPCs can provide dual co-stimulation for the T cells resulting in decreased levels of the markers CD25, TIM3, LAG3, and PD1. This is a less costly and more efficient alternative to peripheral blood mononuclear cells (PBMCs) and cytokine treatments that result in better quality T cell for adoptive transfer back into patients.

In some embodiments, the disclosed aAPCs secrete an antibody (e.g. anti-PD1 or PDL1) that interferes with suppression of T cells, e.g. by ligation of PD1 with PDL1. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders. The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive.

Also disclosed herein are methods of increasing the purity of CAR T cells engineered to express NKG2D and is in a population of immune cells, comprising providing an artificial antigen presenting cell (aAPC) comprising a cell having a membrane, wherein the aAPC expresses ligands for NKG2D (such as for example MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1), and comprises said NKG2D ligands, anti-NKG2D antibodies, and/or anti-NKG2D antibody fragments on its membrane; and incubating the immune cell population with the aAPC for at least 8, 9, 10, 11, 12, 13, 14, 15, 16, 17, 18, 19, 20, 21, 22, 23, 24, 26, 28, 30, 32, 34, 36, or 48 hours; wherein the aAPC induces the CAR T cells to proliferate. These AAPC may also enrich other immune cells that express the NKG2D receptor.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a cartoon showing cell surface ligands for NKG2D.

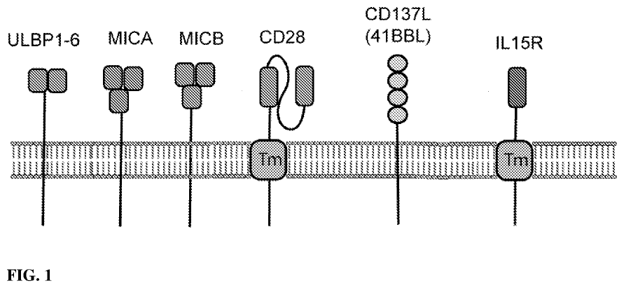

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Methods of Use

Disclosed herein are methods for expanding an immune cell for use in immune therapy. The disclosed methods comprise providing an artificial antigen presenting cell (aAPC) comprising a cell having a membrane, wherein the aAPC expresses natural-killer group 2, member D (NKG2D) ligands and/or antibodies that bind NKG2D (including, but not limited to proteins, such as, for example, F(ab′)2, Fab′, Fab, and/or scFv), and comprises said NKG2D ligands, anti-NKG2D antibodies, and/or anti-NKG2D antibody fragments on its cell membrane.

NKG2D is a major recognition receptor for the detection and elimination of transformed and infected cells as its ligands are induced during cellular stress, either as a result of infection or genomic stress such as in cancer. As used herein, the NKG2D protein can be any mammalian NKG2D protein, but is preferably a human NKG2D protein. The protein sequence for NKG2D is known in the art. For example, in some embodiments, the ectodomain of NKG2D comprises an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity VTRQMCIYTNPTSCNEIYGKFSSAYLACDGKQMEIITLLNPSLISGDEWQWSGNTPIHVLG MWHYSKVLKLLDQDEKSYVKLLSANQSMCSAQSEYWNKSEDFFQYCNNKYCIWNKPCP GCYSETLPIQVEQNFLSNLFVASWI (SEQ ID NO:1), or a fragment thereof of at least 100, 110, 120, 130, 135, 136, 137, 138, 139, 140, 141, 142, or 143 amino acids that can bind the induced-self proteins. All NKG2D ligands are homologous to MHC class I molecules and are divided into two families: MIC and RAET1/ULBP. Human MIC genes are located within the MHC locus and are composed of seven members (MICA-G), of which only MICA and MICB produce functional transcripts. In mice, MIC genes are absent. Among ten known human RAET1/ULBP genes, six encode functional proteins: RAET1E/ULBP4, RAET1G/ULBP5, RAET1H/ULBP2, RAET1/ULBP1, RAET1L/ULBP6, RAET1N/ULBP3. In mice, proteins from orthologous RAET1/ULBP family fall into three subfamilies: Rae-1, H60, and MULT-1. Accordingly, in one aspect, disclosed herein are methods for expanding an immune cell for use in immune therapy, said methods comprising providing to an isolated immune cell an artificial antigen presenting cell (aAPC) comprising a cell having a membrane, wherein the aAPC expresses NKG2D ligands (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or anti-NKG2D antibodies, and comprises said NKG2D ligands, anti-NKG2D antibodies, and/or anti-NKG2D antibody fragments on its membrane. In one aspect, the method further comprises contacting the isolated immune cell with an effective amount of the aAPC to expand the immune cell in an amount effective for immunotherapy. Thus, in one aspect, disclosed herein are methods of methods for expanding an immune cell for use in immune therapy, said methods comprising a) providing to an isolated immune cell an artificial antigen presenting cell (aAPC) comprising a cell having a membrane, wherein the aAPC expresses NKG2D ligands (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or anti-NKG2D antibodies, and comprises said NKG2D ligands, anti-NKG2D antibodies, and/or anti-NKG2D antibody fragments on its membrane; and b) contacting the isolated immune cell with an effective amount of the aAPC to expand the immune cell in an amount effective for immunotherapy.

It is understood and herein contemplated that by incubating the aAPC in an immune cell population, the aAPC will selectively stimulate and induce proliferation of the cells to which they bind (for example, CAR T cells including, but not limited to CAR T cells comprising an NKG2D CAR (NKG2D CAR T cells). As the incubation time increases so too does the purity of the aAPC bound cell population. Thus, in one aspect, disclosed herein are methods of increasing the purity of immune cells (such as, for example, CAR T cells including, but not limited to NKG2D CAR T cells), wherein the immune cells are incubated with the aAPC for at least 8, 9, 10, 11, 12, 13, 14, 15, 16, 17, 18, 19, 20, 21, 22, 23, 24, 26, 28, 30, 32, 34, 36, 48, 60 hours, 3, 4, 5, 6, 7, 10, 14, 18, 21, 28, or 30 days.

It is understood and herein contemplated that the immune cell used in the disclosed methods can be isolated from a subject receiving the immune therapy (an autologous donor source), from a type match donor source (i.e., syngeneic), from a non-type matched donor source of the same species (i.e, an allogeneic source), a donor of a different species (xenogeneic source), or cell line. In one aspect, the immune cell can be a chimeric antigen receptor (CAR) T cell (including, but not limited to CAR T cells comprising an NKG2D CAR such as, for example, CYAD-01 (previously known as NKR-2)), tumor infiltrating lymphocyte (TIL), or marrow-infiltrating lymphocyte (MIL). In some aspects, where the CAR T cell comprises a NKG2D chimeric antigen receptor (CAR), the CAR can comprise a NKG2D ectodomain, and a transmembrane domain. In some aspects, the NKG2D CAR T cell can further comprise an intracellular signaling domain (such as, for example a CD3 zeta (CD3ζ) signaling domain) and/or a co-stimulatory domain (such as, for example, DAP10, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof). A “signaling domain (SD)” generally contains immunoreceptor tyrosine-based 20 activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors. In one aspect, the CAR T cells can comprise a dual CAR T cell containing the disclosed NKG2D CAR, and at least one other CAR with a different ligand binding target, such as CD33, CD123, TIM3, or CLEC12A. In other aspects, the immune cell can comprise a natural killer (NK) cell, a CAR NK cell, an NK-T cell, a cytokine-induced memory NK cell, a cytokine-induced killer (CIK) cell, or a γδ T cell.

In one aspect, it is understood and herein contemplated that the aAPC expressing NKG2D ligands (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) can be further engineered to enhance the aAPC ability to bind T cells and stimulate proliferation. For example, an antibody or antibody fragment (such as, for example, scFv) that binds to the T cell receptor (CD3) or that binds to a co-stimulatory molecule on T cells (for example, CD28 or 4-1BB). Accordingly, in one aspect disclosed herein are methods of methods for expanding an immune cell for use in immune therapy or methods for increasing the purity of CAR T cells said methods comprising providing an aAPC; wherein the aAPC comprises a NKG2D ligand or anti-NKG2D antibody on its cell membrane and wherein the aAPC further comprises one or more scFv or ligands that bind a co-stimulatory molecule on T-cells (such as, for example, CD28 and/or 4-1BB), one or more scFv that selectively bind CD3, and/or a combination thereof on the cell membrane.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, the term “antibody or fragments thereof” encompasses chimeric antibodies and hybrid antibodies, with dual or multiple antigen or epitope specificities, and fragments, such as F(ab′)2, Fab′, Fab, scFv, and the like, including hybrid fragments. Thus, fragments of the antibodies that retain the ability to bind their specific antigens are provided. For example, fragments of antibodies which maintain NKG2D, CD3, CD28, CD137, PD1, CTLA4, LAG3, TIM3, BTLA, CD160, 2B4, A2aR, and KIR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 105 M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

In one aspect, the aAPCs expressing an (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or antibodies that bind NKG2D (including, but not limited to antibody fragments, such as, for example, F(ab′)2, Fab′, Fab, and/or scFv) disclosed herein can secrete or express surface bound NKG2D ligands, NKG2D antibodies, or NKG2D antibody fragments that bind molecules of the T cell inhibitory pathway. In some embodiments, the disclosed aAPCs secrete an antibody or antibody fragment (for example, an scFv) that interferes with suppression of T cells, e.g. by ligation of PD1 with PDL1 such as, for example, use of an anti-PD1 or PDL1 antibody or antibody fragment. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders.

The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive. In one aspect, other immune cell inhibitory molecule comprises CTLA4, LAG3, TIM3, BTLA, CD160, 2B4, A2aR, PD-1, ICOS, CD25, TIM3, LAG3, PD1, CD40, CD137, OX40, CD2, LFA-1, CD28, CD154, BTLA, CD160, TIM 1, TIM 4, KIR, any glucocorticoid-induced tumor necrosis factor-related receptor (GITR), and/or any combination thereof. Thus, in one aspect, disclosed herein are methods for expanding an immune cell isolated from a subject for autologous immune therapy and/or methods for increasing the purity of CAR T cells said methods comprising a) providing an artificial antigen presenting cell (aAPC) comprising a cell membrane; wherein the aAPC expresses an NKG2D ligand (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or antibodies that bind NKG2D (including, but not limited to antibody fragments, such as, for example, F(ab′)2, Fab′, Fab, and/or scFv) and comprises said NKG2D ligands, anti-NKG2D antibodies, and/or anti-NKG2D antibody fragments on its cell membrane; wherein the cell secretes one or more single chain variable fragment (scFv) antibodies that bind a T cell inhibitory molecule, or a combination thereof; and b) contacting the isolated immune cell with an effective amount of the aAPC to expand the immune cell in an amount effective for immunotherapy. In one aspect, the aAPC can further comprise on its membrane: one or more scFv that selectively bind an immune cell selective receptor (such as, for example CD3) and one or more scFv or ligands that bind a co-stimulatory molecule on T-cells (such as, for example, CD28 and/or 4-1BB). For example, in one aspect disclosed herein are methods for expanding an immune cell isolated from a subject for autologous immune therapy, comprising a) providing an artificial antigen presenting cell (aAPC) comprising a cell membrane; wherein the aAPC expresses NKG2D ligand (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or antibodies that bind NKG2D (including, but not limited to antibody fragments, such as, for example, F(ab′)2, Fab′, Fab, and/or scFv) and comprises said NKG2D ligands, anti-NKG2D antibodies, and/or anti-NKG2D antibody fragments on its cell membrane, wherein the cell secretes one or more single chain variable fragment (scFv) antibodies that bind a T cell inhibitory molecule, or a combination thereof, wherein the cell contains on its membrane: one or more scFv that selectively bind CD3 and one or more scFv or ligands that bind CD28 and/or 4-1BB (such as, for example an anti-CD38 scFv and/or 4-BBL); and b) contacting the isolated immune cell with an effective amount of the aAPC to expand the immune cell in an amount effective for immunotherapy.

In one aspect, the methods of expanding immune cells and/or increasing the purity of immune cells can be used for expanding and/or purifying CAR T cells, TILs, or MILs which can be used in immunotherapy. It is understood and herein contemplated that the use of said cells can comprise expanding CAR T cells, TILs, or MILs from a tissue from a subject. In one aspect, the CAR T cells, TILs, or MILs may be obtained from any tissue (such as, for example, biopsy, blood, urine, sputum, saliva, tissue lavage) in a subject by any means known in the art (tissue resection, biopsy phlebotomy, core biopsy). Because the tissue sample can be used, it can be advantageous to screen expanded CAR T cells, TILs, or MILs for desired activity (such as, for example, tumoricidal activity via expression of CD107). Thus, in one aspect, disclosed herein are methods for expanding tumor infiltrating lymphocytes for use in immunotherapy, comprising a) providing an artificial antigen presenting cell (aAPC) comprising a cell membrane; wherein the aAPC expresses NKG2D ligand (such as, for example, MICA, MICB, RAET1E/ULBP4, RAET1G/ULBP5, RAET1H/ULBP2, RAET1/ULBP1, RAET1L/ULBP6, and/or RAET1N/ULBP3 as well as mouse ligands H60, MULT-1, and Rae-1) and/or antibodies that bind NKG2D (including, but not limited to antibody fragments, such as, for example, F(ab′)2, Fab′, Fab, and/or scFv) and comprises said NKG2D ligands, anti-NKG2D antibodies, and/or anti-NKG2D antibody fragments on its cell membrane; b) expanding MILs and/or TILs from a biopsy of a tumor from a subject; c) screening the MILs and/or TILs for tumoricidal activity using flow cytometry to detect CD107 expression; and d) contacting the tumoricidal MILs and/or TILs with an effective amount of the aAPC to expand the tumoricidal MILs and/or TILs. In one aspect, the disclosed methods can further comprise infusing the expanded tumoricidal MILs and/or TILs into the subject in an effective amount to treat the tumor. In one aspect, the disclosed methods can further comprise aAPC wherein the cell secretes one or more single chain variable fragments (scFv) that bind a T cell inhibitory molecule, or a combination thereof, wherein the cell contains on its membrane one or more scFv that selectively bind CD3 and one or more scFv or ligand that binds a co-stimulatory molecule on T-cells.

It is understood and herein contemplated that the expansion of immune cells and/or increasing the purity of immune cells (such as, for example T cells, NK cells, or B cells) including CAR T cells, TILs, and MILs can occur ex vivo, in vitro, or in situ with the expansion occurring outside the subject and administration occurring after expansion. However, it is understood and herein contemplated that the expansion of immune cells including CAR T cells, TILs, and MILs can also occur in vivo by directly administering aAPC comprising an scFv that binds to a T cell inhibitory molecule, and an scFv recognizing an immune cell receptor (such as, for example) CD3 and scFv or ligands binding to co-stimulatory molecules (such as, CD28 and 4-1BB) directly to the subject in need of treatment.

In one aspect, the aAPC can further comprise on its membrane surface expression of a scFv or ligand that specifically binds a cytokine such as, IL2R, IL7R, IL12R, IL15R, IL18R, IL10R, or any combination thereof.

The aAPC can be derived from any antigen presenting cell including a cell line such as, for example K562, NIH/3T3, Chinese hamster ovary (CHO), or Human Embryonic Kidney (HEK) cell line.

It is understood and herein contemplated that the disclosed methods can result in an expanded immune cell. Accordingly, in one aspect disclosed herein are immune cells produced by any method for expanding immune cells disclosed herein.

In one aspect, it is understood and herein contemplated that the immune cells generated by the disclosed methods can be used in an immunotherapy to treat, inhibit, reduce, decrease, ameliorate and/or prevent a cancer and/or metastasis. Accordingly, in one aspect, disclosed herein are methods of treating, inhibiting, reducing, decreasing, ameliorating, and/or preventing a cancer and/or metastasis comprising administering to a subject with a cancer an immune cell expanded by any of the methods disclosed herein. Stated slightly differently, in one aspect, disclosed herein are methods of treating, inhibiting, reducing, decreasing, ameliorating, and/or preventing a cancer and/or metastasis comprising a) providing an aAPC comprising a cell membrane, wherein the aAPC expresses a NKG2D ligand (such as, for example, MICA, MICB, RAET1E/ULBP4, RAET1G/ULBP5, RAET1H/ULBP2, RAET1/ULBP1, RAET1L/ULBP6, RAET1N/ULBP3, H60, MULT-1, and/or Rae-1) or anti-NKG2D antibody or anti-NKG2D antibody fragment thereof and comprises the NKG2D ligand or anti-NKG2D antibody or anti-NKG2D antibody fragment thereof on its membrane; b) contacting an isolated immune cell (such as, for example, a CAR T cell, TIL, MIL, NK cell, NK-T cell, CAR NK cell, a cytokine-induced memory NK cell, a CIK cell, or a γδ T cell) with an effective amount of the aAPC to expand the immune cell in an amount effective for immunotherapy; expanding the immune cell (for example, expanding for at least 9, 10, 11, 12, 13, 14, 15, 16, 17, 18, 19, 20, 21, 22, 23, 24, 26, 28, 30, 32, 34, 36, or 48 hours); and d) administering the immune cell to the subject with a or at risk for developing a the cancer and/or metastasis. In some aspects the aAPC further expresses one or more ligands and/or one or more single chain variable fragment (scFv) antibodies that bind a T cell inhibitory molecule (such as, for example, PD1, PD-L1, CTLA4, LAG3, TIM3, BTLA, CD160, 2B4, A2aR, KIR, or any combination thereof), or a combination thereof. In one aspect, the aAPC further comprises on its cell membrane one or more scFv or ligands that bind a co-stimulatory molecule on T-cells (such as, for example CD28 and/or 4-1BB), one or more scFv that selectively bind CD3, or a combination thereof.

In one aspect, it is understood and herein contemplated that any of expanded immune cells disclosed herein and any of the methods of treating, inhibiting, reducing, decreasing, ameliorating, and/or preventing a cancer and/or metastasis disclosed herein can be used to treat, reduce, inhibit, ameliorate, and/or prevent any disease where uncontrolled cellular proliferation occurs such as cancers and/or metastasis. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

It is understood and herein contemplated that the disclosed methods of treating, preventing, inhibiting, ameliorating, and/or reducing a cancer and/or metastasis in a subject comprising administering any of the expanded immune cells disclosed herein can further comprise the administration of any anti-cancer agent that would further aid in the reduction, inhibition, treatment, and/or elimination of the cancer or metastasis (such as, for example, gemcitabine). Anti-cancer agents that can be used in the disclosed bioresponsive hydrogels or as an additional therapeutic agent in addition to the disclosed pharmaceutical compositions, and/or bioresponsive hydrogel matrixes for the methods of reducing, inhibiting, treating, ameliorating, decreasing, preventing, and/or eliminating a cancer and/or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for expanding an immune cell isolated from a subject for use in immune therapy, comprising
a) providing an artificial antigen presenting cell (aAPC) comprising a cell membrane, wherein the aAPC expresses a natural-killer group 2, member D (NKG2D) ligand or anti-NKG2D antibody or anti-NKG2D antibody fragment thereof and comprises the NKG2D ligand or anti-NKG2D antibody or anti-NKG2D antibody fragment thereof on its membrane; and
b) contacting the isolated immune cell with an effective amount of the aAPC to expand the immune cell in an amount effective for immunotherapy.

2. The method of claim 1, wherein the aAPC comprises an NKG2D ligand, and wherein the NKG2D ligand comprises H60, MULT-1, Rae-1, MICA, MICB, RAET1E/ULBP4, RAET1G/ULBP5, RAET1H/ULBP2, RAET1/ULBP1, RAET1L/ULBP6, and/or RAET1N/ULBP3.

3. (canceled)

4. (canceled)

5. The method of claim 1, wherein the immune cell comprises a chimeric antigen receptor (CAR) T cell, tumor infiltrating lymphocyte (TIL), marrow-infiltrating lymphocyte (MIL), natural killer (NK) cell, an NK-T cell, CAR NK cell, a cytokine-induced memory NK cell, a cytokine-induced killer (CIK) cell, or a 6 T cell.

6. The method of claim 5, wherein the CAR T cell has been engineered to express NKG2D.

7. (canceled)

8. The method of claim 1, wherein the aAPC further expresses one or more single chain variable fragment (scFv) antibodies that bind a T cell inhibitory molecule, or a combination thereof.

9. The method of claim 8, wherein the T cell inhibitory molecule comprises PD1, PDL1, CTLA4, LAG3, TIM3, BTLA, CD160, 2B4, A2aR, KIR, or a combination thereof.

10. (canceled)

11. The method of claim 1, wherein the aAPC contains on its cell membrane one or more scFv or ligands that bind a co-stimulatory molecule on T-cells, one or more scFv that selectively bind CD3, or a combination thereof.

12. The method of claim 11, wherein the co-stimulatory molecule comprises CD28 or 4-1BB.

13. (canceled)

14. An immune cell produced by the method of claim 1.

15. A method of treating a cancer or metastasis in a subject comprising administering to the subject an immune cell of claim 14.

16. A method for increasing the purity of CAR T cells in a population of immune cells, comprising
a) providing an artificial antigen presenting cell (aAPC) comprising a cell membrane, wherein the aAPC expresses a natural-killer group 2, member D (NKG2D) ligand or anti-NKG2D antibody or anti-NKG2D antibody fragment thereof and comprises the NKG2D ligand or anti-NKG2D antibody or anti-NKG2D antibody fragment thereof on its membrane; and
b) incubating the CAR T cell with the aAPC for at least 8 hours; wherein the aAPC induces the CAR T cells to proliferate.

17. The method of claim 16, wherein the aAPC comprises an NKG2D ligand, and wherein the NKG2D ligand comprises H60, MULT-1, Rae-1, MICA, MICB, RAET1E/ULBP4, RAET1G/ULBP5, RAET1H/ULBP2, RAET1/ULBP1, RAET1L/ULBP6, and/or RAET1N/ULBP3.

18. (canceled)

19. (canceled)

20. The method of claim 16, further comprising an immune cell population provided with the CAR T cell population; wherein the immune cell population comprises one or more of the T cell population comprising naïve T cells, activated T cells, memory T cells, γδ T cells, natural killer (NK) cell, an NK-T cell, a cytokine-induced memory NK cell, a cytokine-induced killer (CIK) cell, tumor infiltrating lymphocyte (TIL), and marrow-infiltrating lymphocyte (MIL).

21. The method of claim 16, wherein the CAR T cell has been engineered to express NKG2D.

22. The method of claim 16, wherein the immune cells are incubated with the aAPC for at least 9, 10, 11, 12, 13, 14, 15, 16, 17, 18, 19, 20, 21, 22, 23, 24, 26, 28, 30, 32, 34, 36, or 48 hours.

23. The method of claim 16, wherein the aAPC further expresses one or more single chain variable fragment (scFv) antibodies that bind a T cell inhibitory molecule, or a combination thereof.

24. The method of claim 23, wherein the T cell inhibitory molecule comprises PD1, PDL1, CTLA4, LAG3, TIM3, BTLA, CD160, 2B4, A2aR, KIR, or a combination thereof.

25. (canceled)

26. The method of claim 16, wherein the cell membrane contains on its membrane one or more scFv or ligands that bind a co-stimulatory molecule on T-cells, one or more scFv that selectively bind CD3, or a combination thereof.

27. The method of claim 26, wherein the co-stimulatory molecule comprises CD28 or 4-1BB.

28. (canceled)

29. The method of claim 16, wherein the immune cell population consists essentially of a T cell population.
